(19) United States
(12) Patent Application Publication
(10) Pub. No.: US 2023/0005617 A1
(43) Pub. Date: Jan. 5, 2023

(54) HEALTH SUPPORT APPARATUS, HEALTH SUPPORT METHOD, AND RECORDING MEDIUM RECORDING HEALTH SUPPORT PROGRAM

(71) Applicant: KABUSHIKI KAISHA TOSHIBA, Tokyo (JP)

(72) Inventors: Kenichi DONIWA, Asaka (JP); Takahiro TANAKA, Akishima (JP); Kosuke HARUKI, Tachikawa (JP); Masahiro OZAWA, Yokohama (JP)

(73) Assignee: KABUSHIKI KAISHA TOSHIBA, Tokyo (JP)

(21) Appl. No.: 17/652,133

(22) Filed: Feb. 23, 2022

(30) Foreign Application Priority Data
Jul. 2, 2021 (JP) 2021-110860

(51) Int. Cl. G16H 50/20; G16H 50/30; G16H 50/70; G16H 20/30; G16H 20/60; G16H 10/60

(52) U.S. Cl. CPC G16H 50/20; G16H 50/30; G16H 50/70; G16H 20/30; G16H 20/60; G16H 10/60

(57) ABSTRACT

A health support apparatus includes a processor including hardware. The processor accepts input of a first factor that is directly controlled by improving a lifestyle habit of a person subjected to a medical checkup and a second factor that is not directly controlled by improving a lifestyle habit of the person. The processor generates a lifestyle habit combination pattern. The processor calculates change amounts of the first factor and the second factor corresponding to the combination pattern. The processor predicts a disease risk value representing a disease risk of a disease of the person based on at least the change amount of the second factor.

### CROSS-REFERENCE TO RELATED APPLICATIONS

This application is based upon and claims the benefit of priority from the Japanese Patent Application No. 2021-110860, filed Jul. 2, 2021, the entire contents of which are incorporated herein by reference.

### FIELD

Embodiments described herein relate generally to a health support apparatus, a health support method, and a recording medium recording a health support program.

### BACKGROUND

Precision medicine has recently been proposed. The precision medicine is a medicine that analyzes medical treatment methods at the individual level and selects an optimal medical treatment method from the analyzed medical treatment methods. In health guidance against lifestyle-related diseases, optimal goal setting at the individual level is desired as is the case with precision medicine. For example, in order to reduce the diabetic risk, health guidance is desired to be provided in accordance with individual health conditions such that a given person is instructed to achieve 7% weight loss and another person is instructed to achieve 3% weight loss instead of uniformly instructing all persons to achieve 5% weight loss.

### BRIEF DESCRIPTION OF THE DRAWINGS

FIG. 1 is a block diagram showing the arrangement of an example of a health support apparatus according to the first embodiment;

FIG. 2 is a view showing an example of a lifestyle habit improvement combination pattern;

FIG. 3A is a conceptual diagram showing the machine learning of a factor change prediction model as the first example;

FIG. 3B is a conceptual diagram showing the machine learning of a factor change prediction model as the second example;

FIG. 3C is a conceptual diagram showing the machine learning of a factor change prediction model as the third example;

FIG. 4 is a block diagram showing an example of the hardware arrangement of the health support apparatus;

FIG. 5 is a flowchart showing the operation of the health support apparatus according to the first embodiment;

FIG. 6 is a block diagram showing the arrangement of an example of a factor change calculation unit according to the second embodiment;

FIG. 7A is a view showing an example of a 40's male weight change ratio table;

FIG. 7B is a view showing an example of a 60's male weight change ratio table;

FIG. BA is a view showing an example of a 40's male second factor change table;

FIG. 8B is a view showing an example of a 60's male second factor change table; and

FIG. 9 is a flowchart showing the operation of a health support apparatus according to the second embodiment.

### DETAILED DESCRIPTION

In general, according to one embodiment, a health support apparatus includes a processor including hardware. The processor accepts input of a first factor that is directly controlled by improving a lifestyle habit of a person subjected to a medical checkup and a second factor that is not directly controlled by improving a lifestyle habit of the person. The processor generates a lifestyle habit combination pattern. The processor calculates change amounts of the first factor and the second factor corresponding to the combination pattern. The processor predicts a disease risk value representing a disease risk of a disease of the person based on at least the change amount of the second factor.

Embodiments will be described below with reference to the accompanying drawings.

### First Embodiment

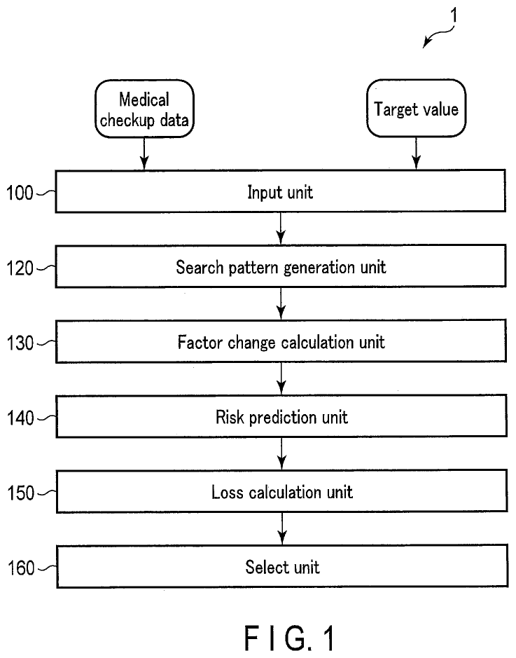

FIG. 1 is a block diagram showing the arrangement of an example of a health support, apparatus according to the first embodiment. A health support apparatus 1 includes an input unit 100, a search pattern generation unit 120, a factor change calculation unit 130, a risk prediction unit 140, a loss calculation unit 150, and a select unit 160.

The input unit 100 accepts the input of medical checkup data and a target value. For example, the input unit 100 may be configured to accept the input of medical checkup data and a target value by the operation of the health support apparatus 1 by the user. In this case, the “user” is a person who uses the health support apparatus 1. The user may be a person subjected to a medical checkup or a healthcare professional such as a doctor. In addition, the input unit 100 may be configured to accept medical checkup data stored in a storage medium outside the health support apparatus 1 (not shown) via a communication medium. Furthermore, the input unit 100 may be configured to accept medical checkup data transferred from the storage medium in response to when the storage medium is mounted in the health support apparatus 1.

In this case, medical checkup data according to this embodiment includes the inspection value of a factor used for the prediction of the risk of a lifestyle-related disease. Such factors include first factors and second factors. The first factors are factors that can be directly controlled by improving the lifestyle habit of a person subjected to a medical checkup. For example, the weight of the person is directly changed by executing exercise and improving the diet. Accordingly, the weight is included in the first factors. In addition, the body fat percentage and the like are included in the first factors. In contrast, the second factors are factors that cannot be directly controlled by improving the lifestyle habit of the person subjected to the medical checkup. For example, HbA1c (hemoglobin A1c) is not directly changed by executing exercise and improving the diet. Accordingly, HbA1c is included in the second factors. In addition, various biopsy values such as GOT (Glutamic Oxaloacetic Transaminase), LDL (Low Density Lipoprotein), and a blood pressure are included in the second factors. Note that HbA1c and the like are factors that cannot be directly controlled by improving the lifestyle habit but can be indirectly changed by a change in weight or the like accompanying the improvement of the lifestyle habit. That is, the second factors include factors that are indirectly controlled by changes in the first factors.

In addition, a target value indicates, for example, to which degree the disease risk value of a specific disease such as a lifestyle-related disease is to be reduced. A target value may be designated by a relative value or absolute value. In this case, the “disease risk value” is the onset probability of a specific disease in a given period. For example, when the disease risk of diabetes is to be reduced from 30% to 20%, the risk reduction target value (relative value) is 10(%) when being designated by a relative value, whereas the risk reduction target value (absolute value) is 20(%) when being designated by an absolute value. Alternatively, a target value may be the one for a change in any of factors such as weight included in medical checkup data. A target value fora change in factor may be designated by an absolute value or relative value like a disease risk value reduction target value.

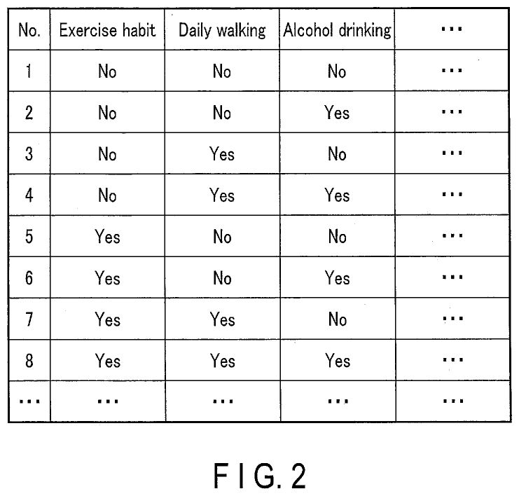

The search pattern generation unit 120 generates a lifestyle habit improvement search pattern. A lifestyle habit improvement search pattern includes a plurality of lifestyle habit combination patterns. FIG. 2 shows an example of lifestyle habit improvement combination patterns. The example shown in FIG. 2 indicates at least three items, namely “exercise habit”, “daily walking”, and “alcohol drinking” as lifestyle habits. The search pattern generation unit 120 generates a lifestyle habit improvement combination pattern by, for example, setting “yes” and “no” of these lifestyle habits. For example, setting “yes” for “exercise habit” and “daily walking” indicates that the lifestyle habits will be improved. On the other hand, setting “no” for “alcohol drinking” indicates that the lifestyle habit will be improved. For example, in pattern “No. 1”, “exercise habit”, “daily walking”, and “alcohol drinking” each are set to “no”. Pattern “No. 1” having such setting indicates that avoiding “alcohol drinking” will improve the lifestyle habits. In addition, in pattern “No. 2”, “exercise habit” and “daily walking” are set to “no”, and “alcohol drinking” is set to “yes”. Pattern “No. 2” having such setting indicates that the lifestyle habits will not be improved concerning “exercise habit”, “daily walking”, and “alcohol drinking”. In this case, “no” in FIG. 2 does not indicate that “exercise habit”, “daily walking”, and “alcohol drinking” are not practiced at all. Note that “no” in FIG. 2 may include practicing “exercise habit”, “daily walking”, and “alcohol drinking” to such a degree that no influence is imposed on the prediction of a disease risk value. In addition, referring to FIG. 2, setting “yes” and “no” concerning the lifestyle habits will generate a lifestyle habit improvement pattern. In contrast to this, a lifestyle habit combination pattern may be generated in additional consideration of the amounts of the respective lifestyle habits. For example, “yes” for exercise habit may be divided into three patterns, namely, “little”, “intermediate”, and “much”.

In this case, the lifestyle habit data used for the generation of a lifestyle habit search pattern by the search pattern generation unit 120 is stored in, for example, the search pattern generation unit 120. Alternatively, the lifestyle habit data may be stored in a storage medium outside the health support apparatus 1. In this case, the search pattern generation unit 120 acquires lifestyle habit data from the external storage medium as needed.

The factor change calculation unit 130 calculates the change amount of each factor included in the medical checkup data based on the lifestyle habit search pattern generated by the search pattern generation unit 120. The factor change calculation unit 130 predicts the change amount of factor based on, for example, a factor change amount prediction model. This factor change prediction model is, for example, a machine learning model configured to receive medical checkup data and a search pattern and output the change amount of each factor.

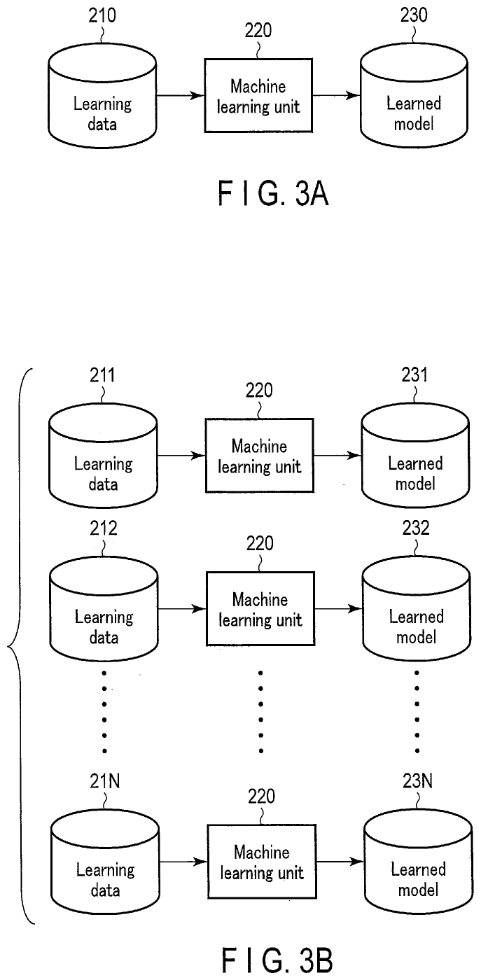

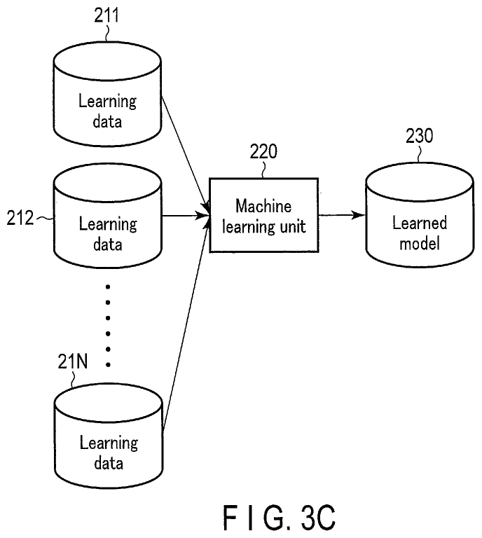

An example of a learning method for a factor change prediction model will be described. FIG. 3A is a conceptual diagram of machine learning of the factor change prediction model as the first example. As shown in FIG. 3A, machine learning of the factor change prediction model is executed when learning data 210 is input to a machine learning unit 220. Executing learning using a predetermined amount of learning data 210 will generate a learned model 230 as a factor change prediction model.

The learning data 210 includes feature amount data and teacher data. The feature amount data is the data of feature amounts that can be used for the prediction of a change in factor, more specifically, feature amounts such as gender, age, weight, biopsy value, and lifestyle habit. Biopsy values include various types of biopsy values including first and second factors such as blood pressure, GOT, and LDL. Lifestyle habits include Yes or No or amounts of “exercise habit”, “daily walking”, and “alcohol drinking” described above. Teacher data is the actual change amount of a prediction target factor based on a related feature amount.

Using the learning data 210, the machine learning unit 220 learns the relationship between a lifestyle habit combination pattern and the change amount of a factor which changes in accordance with the pattern. The machine learning algorithm in the machine learning unit 220 may be an arbitrary machine learning algorithm such as neural network, decision tree, or linear regression, which allows learning of the relationship between a feature amount and the change amount of a factor. The machine learning unit 220 makes the factor change calculation unit 130 store the learned model 230 generated as a result of a predetermined amount of learning as a factor change prediction model. The machine learning unit 220 can be implemented by a computer including a processor and a memory. For example, Kazuyo Tsushita, “Practice and Evaluation of Lifestyle Intervention on Program”, The Journal of the Japanese Society of Internal Medicine/Volume 105/Issue 9, [Online] [Search: Apr. 28, 2021], Internet URL: https://www.jstage.jst.go.jp/article/naika/105/9/105_1654/article/-char/ja/reports that as the weight changes accompanying an improvement in lifestyle habit, a biopsy value such as GOT, HbA1c, or LDL as a second factor significantly changes. That is, improving the lifestyle habit will change a first factor such as weight and biopsy values as second factors such as GOT, HbA1c, and LDL at given ratios. Accordingly, inputting a lifestyle habit search pattern and the values of the respective factors before an improvement in lifestyle habit to this factor change prediction model will make the factor change prediction model output a disease the values of the respective factors that change accompanying the improvement in lifestyle habit. When, for example, the machine learning algorithm is a neural network, the parameters of the neural network, such as a weight coefficient and a bias, are trained such that inputting a lifestyle habit combination pattern and the values of the respective factors before an improvement in lifestyle habit to the neural network will make it output the values of the respective factors that change accompanying the improvement in lifestyle habit. More specifically, the parameters are trained so as to minimize the error between the predicted change amount obtained by the forward propagation of the value of each factor included in the feature amount data to the neural network and the actual change amount included in the teacher data. At this time, the parameters may be trained by inputting a lifestyle habit combination pattern, together with feature amount data, to the neural network or the parameters of the neural network may be trained for each lifestyle habit combination pattern.

A factor change prediction model can be generated for each group such as each gender group or each age group. In this case, learning is executed by using learning data collected for each group. FIG. 3B is a conceptual diagram showing the machine learning of a factor change prediction model as the second example.

Referring to FIG. 3B, stratified learning data are represented by N (N is a natural number) learning data, namely, learning data 211, learning data 212, . . . , learning data 21N. The learning data 211, the learning data 212, . . . , the learning data 21N each include feature amount data similar to that of the learning data 210. Note, however, that the learning data 211, the learning data 212, . . . , the learning data 21N are stratified according to a feature amount as a stratification target. When, for example, the learning data 211, the learning data 212, . . . , the learning data 21N are stratified according to gender, each data group has commonality in terms of gender. For example, the learning data 211 can be a male learning data group for male as gender. The learning data 212 can be a female learning data group as female as gender. Likewise, when the learning data 211, the learning data 212, . . . , the learning data 21N are stratified according to age groups, each data group is learning data based on ages included in the same age group. For example, the learning data 211 can be learning data in 40's as an age group. The learning data 212 can be learning data in 60's as an age group.

The machine learning unit 220 executes learning by individually using each of the learning data 211, 212, . . . , 21N. The machine learning unit 220 makes the factor change calculation unit 130 store learned models 231, 232, . . . , 23N generated as a result of a predetermined amount of learning as factor change prediction models. Each learned model is obtained by learning using stratified learning data. Accordingly, each learning data allows more learning of the change amount of a factor on the corresponding group. The factor change calculation unit 130 predicts the change amount of the factor by selecting a learned model for a group corresponding to the input medical checkup data from the learned models 231, 232, . . . , 23N.

FIG. 3C is a conceptual diagram showing the machine learning of a factor change prediction model as the third example. The third example is an example of stratification with one learned model.

Referring to FIG. 3C, the stratified learning data are represented by the learning data 211, the learning data 212, . . . , the learning data 21N. The learning data 211, the learning data 212, . . . , the learning data 21N are learning data stratified in the same manner as the second example. Note, however, that the third example has identifiers added to feature amount data to allow the discrimination of stratified and collected learning data. The identifiers may be identification numbers or the like. For example, the identification number 1 is added to the feature amount data of the learning data 211, and the identification number 2 is added to the feature amount data of the learning data 212.

The machine learning unit 220 executes learning by using each of the learning data 211, the learning data 212, . . . , the learning data 21N. The machine learning unit 220 makes the factor change calculation unit 130 store the learned model 230 generated as a result of a predetermined amount of learning as a factor change prediction model. The factor change calculation unit 130 predicts the change amount of the factor by inputting the feature amount data including the same identification number as that of the input medical checkup data to the learned model.

The risk prediction unit 140 predicts the disease risk value of a disease for each search pattern based on the change amount of the factor predicted by the factor change calculation unit 130. The risk prediction unit 140 predicts, for example, the disease risk value of the disease designated by the user based on, for example, a disease risk prediction model. This risk prediction model is a machine learning model configured to receive, for example, medical checkup data and output a disease risk value for each disease. As a disease risk prediction method, a generally known arbitrary prediction method can be used. As described above, as a lifestyle habit is improved, a first factor such as weight and biopsy values as second factors such as GOT, HbA1c, and LDL change at given ratios. Accordingly, inputting the values of first and second factors after changes accompanying an improvement in lifestyle habit to this risk prediction model will make the risk prediction model output a disease risk value that changes accompanying the improvement in lifestyle habit.

The loss calculation unit 150 calculates a third loss Loss3 used for selection by the select unit 160 according to equation (1):

Loss3=Loss1+α×Loss2 (1)

where Loss1 is a first loss based on the difference between a disease risk value predicted by the risk prediction unit 140 and a target value corresponding to the disease risk value, Loss2 is a second loss caused by changing a lifestyle habit used for prediction and each corresponding factor, that is, an improvement in lifestyle habit and a factor accompanying the improvement, and a is a parameter for adjusting the weights of the first Loss1 and the second loss Loss2.

The loss calculation unit 150 calculates the first loss Loss1 according to equations (2) given below:

a) If predicted disease risk value risk reduction target value, then

Loss1=0

b) If predicted disease risk value>risk reduction target value, then

Loss1=(predicted disease risk value−risk reduction target value)² (2)

In addition, the loss calculation unit 150 calculates the second loss Loss2 according to equation (3) given below:

Loss2=Σ(Xi−Xi_org)²/Num_X (3)

where Xi is a candidate for the target value for the ith (i is a natural number) input value of a prediction model. In addition, Xi_org is an actual inspection value of the ith input value of the prediction model. Furthermore, Num_X is the number of input values. Note that the respective input values can greatly differ in scale. In this case, the value of Loss2 tends to be influenced by an input value with a large scale. In order to suppress the influence of such a specific input value, the values of Xi and Xi_org may be standardized within the range from 0 to 1.

When the input unit 100 accepts the target value of a change in a factor included in the medical checkup data, the loss calculation unit 150 may calculate Loss1 according to equation (4) given below:

Loss1=(F1−F2)² (4)

where F1 is the change ratio of a factor required to achieve the input factor change target value and F2 is the change ratio of a factor that can be achieved by improving a lifestyle habit. If, for example, an input factor change target value is a target value for GOT, F1 can be the change ratio of the weight required to achieve the GOT change target value and F2 can be the change ratio of the weight that can be achieved by improving a lifestyle habit.

The select unit 160 selects one or more lifestyle habit combination patterns by using the loss calculated by the loss calculation unit 150. For example, when a loss is calculated according to equation (1), a smaller value of the third loss Loss3 indicates that the disease risk value improved by the corresponding lifestyle habit combination pattern is close to the risk reduction target value or the biopsy value of the factor improved by the lifestyle habit combination pattern is closed to the target value. Therefore, for example, when a loss is calculated according to equation (1), the select unit 160 selects one or more lifestyle habit combination patterns from generated search patterns in ascending order of the value of the third loss Loss3. Depending on the calculation of losses, a larger loss value may indicate that the disease risk value improved by the corresponding lifestyle habit combination pattern is closer to the risk reduction target value or the biopsy value of the factor improved by the lifestyle habit combination pattern is close to the target value. In such a case, the select unit 160 selects one or more lifestyle habit combination patterns from generated search patterns in descending order of loss value.

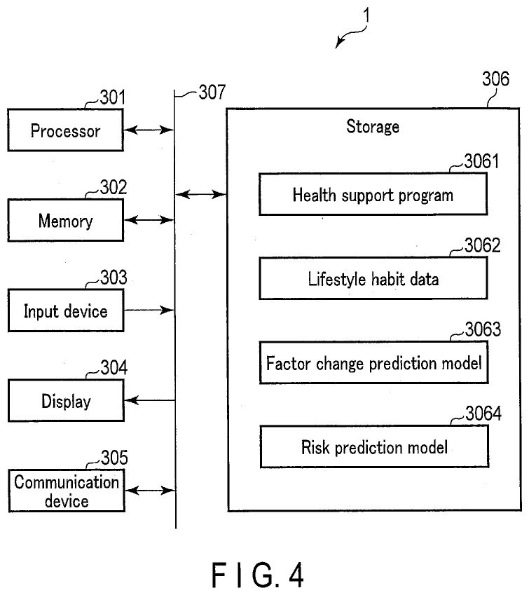

FIG. 4 shows an example of the hardware arrangement of the health support apparatus 1. The health support apparatus 1 includes, as hardware, for example, a processor 301, a memory 302, an input device 303, a display 304, a communication device 305, and a storage 306. The processor 301, the memory 302, the input device 303, the display 304, the communication device 305, and the storage 306 are connected to a bus 307. The health support apparatus 1 may be a terminal apparatus such as a personal computer (PC), smartphone, or tablet terminal.

The processor 301 is a processor that controls the comprehensive operation of the health support apparatus 1. The processor 301 operates as the input unit 100, the search pattern generation unit 120, the factor change calculation unit 130, the risk prediction unit 140, the loss calculation unit 150, and the select unit 160 by executing the health support program stored in, for example, the storage 306. The processor 301 is, for example, a CPU. The processor 301 may be an MPU, GPU, ASIC, FPGA, or the like. The processor 301 may be a single CPU or the like or a plurality of CPUs or the like.

The memory 302 includes a ROM and a RAM. The ROM is a nonvolatile memory. The ROM stores a boot program and the like for the health support apparatus 1. The RAM is a volatile memory. The RAM is used as a work memory when, for example, the processor 301 perform processing.

The input device 303 is an input device including a touch panel, a keyboard, and a mouse. When the input device 303 is operated, a signal corresponding to the operation is input to the processor 301 via the bus 307. The processor 301 performs various types of processing in accordance with such signals. The input device 303 can be used to input, for example, medical checkup data and a risk reduction target value.

The display 304 is a display such as a liquid display or organic EL display. The display 304 displays various types of images.

The communication device 305 is a communication device for allowing the health support apparatus 1 to communicate with an external device. The communication device 305 may be a communication device for wired communication or a communication device for wireless communication.

The storage 306 is a storage such as a flash memory, hard disk drive, or solid state drive. The storage 306 stores various types of programs executed by the processor 301, such as a health support program 3061. The storage 306 also stores lifestyle habit data 3062 for generating a lifestyle habit search pattern. The lifestyle habit data 3062 is, for example, an ID assigned to each lifestyle habit. In addition, the storage 306 stores a factor change prediction model 3063. The storage 306 also stores a risk prediction model 3064 used for the prediction of a disease risk value. The lifestyle habit data 3062, the factor change prediction model 3063, and the risk prediction model 3064 need not always be stored in the storage 306. For example, the lifestyle habit data 3062, the factor change prediction model 3063, and the risk prediction model 3064 may be stored in a server outside the health support apparatus 1. In this case, the health support apparatus 1 acquires necessary information by accessing the server by using the communication device 305.

The bus 307 is a data transfer path for exchanging data among the processor 301, the memory 302, the input device 303, the display 304, the communication device 305, and the storage 306.

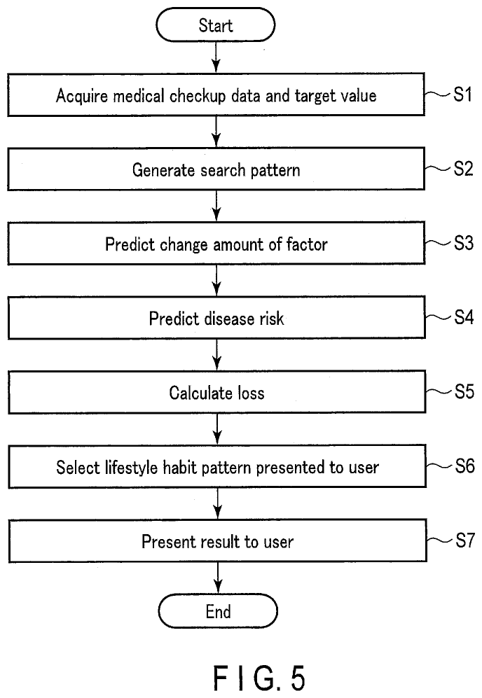

The operation of the health support apparatus 1 according to the first embodiment will be described next. FIG. 5 is a flowchart showing the operation of the health support apparatus 1 according to the first embodiment. The processing in FIG. 5 is executed by the processor 301.

In step S1, the processor 301 acquires medical checkup data and a target value. The medical checkup data may be input via the operation of the input device 303 by the user on the GUI (Graphical User Interface) displayed on the display 304 or input via a storage medium outside the health support apparatus 1. The target value may be input via the operation of the input device 303 by the user on the GUI displayed on the display 304.

In step S2, the processor 301 generates a lifestyle habit search pattern by referring to the lifestyle habit data 3062. The processor 301 may generate a search pattern based on all lifestyle habit combinations generated from the lifestyle habit data 3062 or generate a search pattern based on some lifestyle habit combinations.

In step S3, the processor 301 calculates the change amount of a factor used for the prediction of a disease risk for each search pattern. For example, the processor 301 predicts the change amount of a factor by inputting a search pattern and medical checkup data to the factor change prediction model.

In step S4, the processor 301 predicts a disease risk value. For example, the processor 301 predicts a disease risk value for each search pattern by inputting the value of the factor after the change to a disease risk prediction model.

In step S5, the processor 301 executes loss calculation. For example, the processor 301 calculates losses according to equations (1), (2), and (3) or equations (1), (2), and (4).

In step S6, the processor 301 selects a lifestyle habit combination pattern to be presented to the user. For example, the processor 301 selects one or more lifestyle habit combination patterns in ascending order of the values of losses.

In step S7, the processor 301 presents the result to the user. Thereafter, the processor 301 terminates the processing in FIG. 5. For example, the processor 301 displays the selected lifestyle habit combination pattern and a disease risk value predicted from the corresponding lifestyle habits on the screen of the display 304. A reduction value of the disease risk value or the reduction target value of a factor such as weight and the value of a factor improved by a lifestyle habit may be displayed instead of a disease risk value.

As described above, according to the first embodiment, a machine learning model is used to predict the change amounts of the first factor that can be directly controlled by improving the lifestyle habit of a person subjected to a medical checkup in accordance with a lifestyle habit combination pattern of the person subjected to the medical checkup and the second factor that cannot be directly controlled by improving the lifestyle habit of the person subjected to the medical checkup. A disease risk value can be predicted based on the first and second factors. For example, the weight is reduced over a long period of time, such as after half a year or one year, by improving a lifestyle habit, and the biopsy value is improved by a long-term reduction in weight. According to the first embodiment, a disease risk value can be calculated in consideration of such a long-term reduction in weight and a long-term change in biopsy value. In this manner, in the first embodiment, the second factor that cannot be directly controlled by improving the lifestyle habit of a person subjected to a medical checkup is properly reflected in a prediction result in the prediction of a disease risk value, and hence optimal goal setting at the individual level can be implemented in health guidance.

Stratifying learning data used for the prediction of changes in factors makes it possible to predict the change amounts of the first and second factors of a person subjected to a medical checkup at the individual level. This can provide the person subjected to a medical checkup with more appropriate guidance.

### Second Embodiment

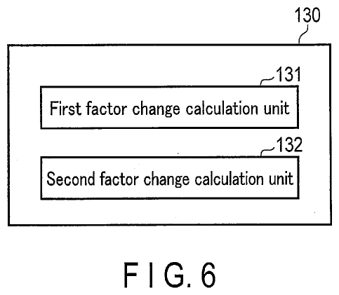

The second embodiment will be described next. A factor change calculation unit 130 according to the second embodiment calculates the change amount of each factor included in medical checkup data based on the lifestyle habit search pattern generated by a search pattern generation unit 120. FIG. 6 shows the arrangement of an example of the factor change calculation unit 130 according to the second embodiment. As shown in FIG. 6, the factor change calculation unit 130 may include a first factor change calculation unit 131 and a second factor change calculation unit 132.

The first factor change calculation unit 131 calculates the change amount of the first factor based on the lifestyle habit search pattern. The second factor change calculation unit 132 calculates the change amount of the second factor. When a change in the first factor influences a change in the second factor, the second factor change calculation unit 132 calculates the change amount of the second factor based on the change amount of the first factor.

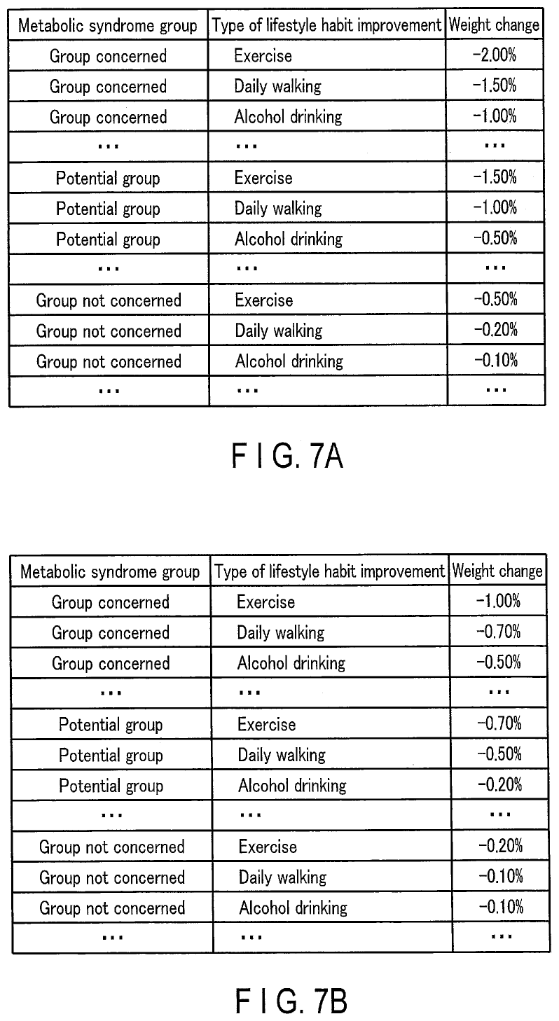

FIGS. 7A and 7B are views each showing an example of a weight change ratio table as an example of a first factor change table. A weight change ratio is a value representing a weight change by percentage from the original weight. A weight change ratio table is a table representing a weight change ratio accompanying a lifestyle habit improvement. In the second embodiment, a weight change ratio table is prepared for each group. FIG. 7A shows an example of a 40's male weight change ratio table. FIG. 7B shows an example of a 60's male weight change ratio table. A weight change ratio table can be generated by actually measuring changes in weight accompanying lifestyle habit improvements with respect to many persons in age groups and gender groups. Obviously, weight change ratio tables in other age groups and weight change ratio tables for females may be further prepared.

Referring to FIGS. 7A and 7B, weight change ratios are divided into three groups, namely, a metabolic syndrome group, a potential metabolic syndrome group, and a non-metabolic syndrome group. The first factor change calculation unit 131 calculates a weight change ratio concerning a first factor by using the weight change ratio table and calculates the change amount of weight based on the calculated weight change ratio. The change amount of weight is, for example, the product of the weight of a person subjected to a medical checkup and a weight change ratio. In this case, the weight change ratio table may include weight change ratios corresponding to the amounts of lifestyle habits.

The tables in FIGS. 7A and 7B can be used as tables for calculating weight change ratios. Tables similar to those shown in FIGS. 7A and 7B are prepared for the respective types of first factors. The first factor change calculation unit 131 calculates the change amount of each first factor by using a first factor change table for a corresponding group.

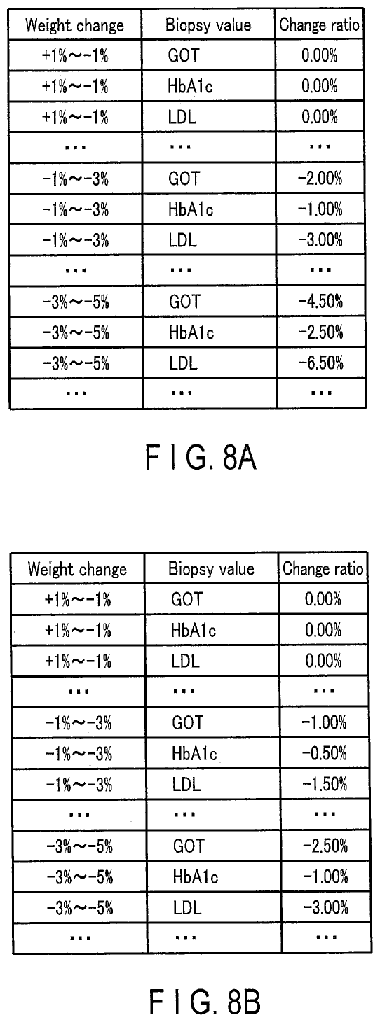

FIGS. 8A and 8B each are an example of a second factor change table. FIGS. 8A and 8B each show a table representing the relationship between weight change ratios and second factor change ratios. In the second embodiment, second factor change tables are prepared for the respective groups. FIG. 8A shows an example of a second factor change table for 40's males. FIG. 8B shows an example of a second factor change table for 60's males. A second factor change table can be generated by actually measuring changes in biopsy value accompanying weight changes with respect to, for example, many persons in age groups and gender groups. Obviously, second factor change tables in other age groups and second factor change tables for age groups of females may be prepared.

The second factor change calculation unit 132 calculates the change amounts of second factors based on the change amount of a first factor for each lifestyle habit search pattern calculated by the first factor change calculation unit 131. For example, referring to FIG. 8A, when the weight change amount is from −1% to +1%, the change ratios of GOT, HbA1c, and LDL each are 0.00%. In contrast to this, when the weight change amount is from −1% to −3%, the change ratios of GOT, HbA1c, and LDL are respectively −2.00%, −1.00%, and −3.00%.

The tables in FIGS. 8A and 8B can be used as tables for the calculation of the change amounts of the second factors from weight change ratios. Tables similar to those shown in FIGS. 8A and 8B are prepared for the respective types of first factors. The second factor change calculation unit 132 calculates the change amount of each second factor by using a second factor change table for a corresponding group.

In addition, the factor change calculation unit 130 calculates the change amount of a factor in accordance with the lifestyle habit search pattern generated by the search pattern generation unit 120. The factor change calculation unit 130 may totalize change ratios for the respective individual improvement patterns of lifestyle habits such as exercise and daily walking or may calculate a change amount by combining a plurality of lifestyle habits.

As the hardware arrangement of the health support apparatus 1, the arrangement shown in FIG. 4 can be basically used. Note, however, that a storage 306 can store first factor change tables and second factor change tables. The first factor change tables and the second factor change tables need not always be stored in the storage 306.

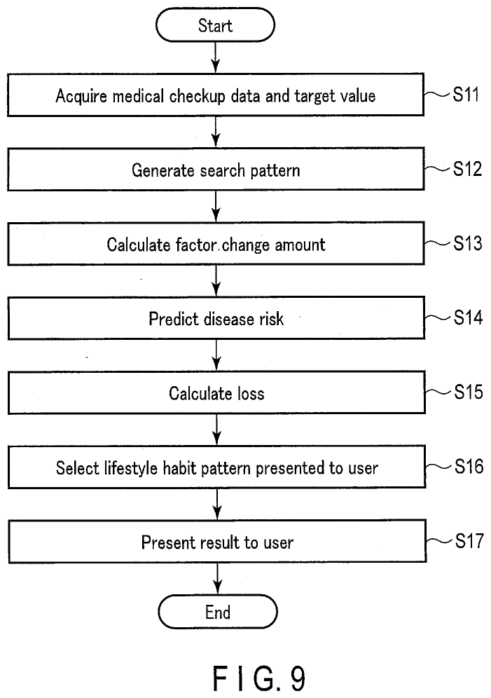

The operation of the health support apparatus 1 according to the second embodiment will be described next. FIG. 9 is a flowchart showing the operation of the health support apparatus 1 according to the second embodiment. The processing in FIG. 9 is executed by a processor 301.

In step S11, the processor 301 acquires medical checkup data and a target value. The medical checkup data may be input, for example, via the operation of an input device 303 by the user on the GUI displayed on a display 304 or may be input via a storage medium outside the health support apparatus 1. The target value may be input via the operation of the input device 303 by the user on the GUI displayed on the display 304.

In step S12, the processor 301 generates a lifestyle habit search pattern by referring to lifestyle habit data 3062. The processor 301 may generate a search pattern by combining all or some of the lifestyle habits that can be generated from the lifestyle habit data 3062.

In step S13, the processor 301 calculates the change amount of a factor used for the prediction of a disease risk for each search pattern. When a change in first factor influences a change in second factor, the processor 301 calculates the change amount of the first factor first by using a first factor change table and then calculates the change amount of the second factor by using a second factor change table.

In step S14, the processor 301 predicts a disease risk value. For example, the processor 301 inputs the value of the factor after the change to a disease risk prediction model and predicts a disease risk value for each search pattern.

In step S15, the processor 301 executes loss calculation. For example, the processor 301 calculates losses according to equations (1), (2), and (3) or (1), (2), and (4).

In step S16, the processor 301 selects a lifestyle habit combination pattern presented to the user. For example, the processor 301 selects one or more lifestyle habit combination patterns in ascending order of the values of losses.

In step S17, the processor 301 presents the result to the user. The processor 301 then terminates the processing in FIG. 9. For example, the processor 301 displays the selected lifestyle habit combination pattern and the disease risk value predicted in the lifestyle habits on the screen of the display 304. A reduction value of the disease risk value may be displayed instead of a disease risk value or the reduction target value of a factor such as weight and the biopsy value of the factor improved by a lifestyle habit may be displayed.

As described above, the second embodiment is configured to calculate, based on tables, the change amounts of the first factor that can be directly controlled by improving the lifestyle habit of a person subjected to a medical checkup in accordance with a lifestyle habit combination pattern of the person subjected to the medical checkup and the second factor that cannot be directly controlled by improving the lifestyle habit of the person subjected to the medical checkup. According to the second embodiment, a disease risk value can be predicted based on the first and second factors. In this manner, as in the first embodiment, in the second embodiment, the second factor that cannot be directly controlled by improving the lifestyle habit of a person subjected to a medical checkup is properly reflected in a prediction result in the prediction of a disease risk value, and hence optimal goal setting at the individual level can be implemented in health guidance.

The tables used for the calculation of changes in factor are prepared for the respective groups such as gender groups and age groups. Accordingly, the change amounts of factors can be calculated in accordance with lifestyle habit combination patterns including differences in gender and age among persons subjected to medical checkups. This makes it possible to use the change amounts of factors for the respective groups based on differences in gender, age, and the like for risk prediction in the second embodiment as in the first embodiment, thereby providing a person subjected to a medical checkup with proper guidance.

[Modification]

The first and second embodiments each have exemplified the stratification based on differences in gender and the stratification based on differences in age. However, stratification may be performed in a different manner. For example, stratification may be performed depending on whether a certain drug is administered. In addition, stratification may be performed based on BMI (Body Mass Index) values. Furthermore, stratification may be performed based on dietary habits such as easing a lot of meats, not eating a lot of meats, or vegetarianism. Moreover, stratification may be performed in an arbitrary manner such as stratification based on whether there is any obese person in a family. These stratifications may be singly used or in combination.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A health support apparatus comprising a processor including hardware and configured to
accept input of a first factor that is directly controlled by improving a lifestyle habit of a person subjected to a medical checkup and a second factor that is not directly controlled by improving a lifestyle habit of the person,
generate a lifestyle habit combination pattern,
calculate change amounts of the first factor and the second factor corresponding to the combination pattern, and
predict a disease risk value representing a disease risk of a disease of the person based on at least the change amount of the second factor.

2. The apparatus according to claim 1, wherein the second factor changes with a change in the first factor, and
the processor calculates a change amount of the first factor and calculates a change amount of the second factor based on the change amount of the first factor.

3. The apparatus according to claim 1, wherein the processor calculates change amounts of the first factor and the second factor by inputting the lifestyle habit combination pattern, a value of the first factor of the person subjected to a medical checkup, and a value of the second factor of the person to a factor change prediction model having learned a relationship between the lifestyle habit combination pattern and change amounts of the first factor and the second factor.

4. The apparatus according to claim 1, wherein the processor calculates change amounts of the first factor and the second factor by inputting the lifestyle habit combination pattern, a value of the first factor of the person subjected to a medical checkup, and a value of the second factor of the person to a corresponding factor change prediction model of a plurality of factor change prediction models each having learned a relationship between the lifestyle habit combination pattern and change amounts of the first factor and the second factor for each group.

5. The apparatus according to claim 1, wherein the processor calculates change amounts of the first factor and the second factor by inputting the lifestyle habit combination pattern, a value of the first factor of the person subjected to a medical checkup to which an identifier of a corresponding group is attached, and a value of the second factor of the person to which an identifier of a corresponding group is attached to a factor change prediction model having learned a relationship between the lifestyle habit combination pattern and change amounts of the first factor and the second factor for each group.

6. The apparatus according to claim 1, wherein the processor calculates change amounts of the first factor and the second factor for each group based on a first factor change table on which a relationship between the lifestyle habit combination pattern and a change amount of the first factor is recorded for each group and a second factor change table on which a relationship between a change amount of the first factor and a change amount of the second factor is recorded for each group.

7. The apparatus according to claim 4, wherein the processor calculates change amounts of the first factor and the second factor upon classifying the person subjected to a medical checkup according to gender.

8. The apparatus according to claim 4, wherein the processor calculates change amounts of the first factor and the second factor upon classifying the person subjected to a medical checkup according to age.

9. The apparatus according to claim 4, wherein the processor calculates change amounts of the first factor and the second factor upon classifying the person subjected to a medical checkup according to whether a drug is administered.

10. The apparatus according to claim 4, wherein the processor calculates change amounts of the first factor and the second factor upon classifying the person subjected to a medical checkup according to a dietary habit of the person.

11. The apparatus according to claim 4, wherein the processor calculates change amounts of the first factor and the second factor upon classifying the person subjected to a medical checkup according to whether there is any obese person in a family of the person.

12. The apparatus according to claim 1, wherein the first factor includes a weight of the person subjected to a medical checkup, and the second factor includes a biopsy value of the person.

13. A health support method comprising:
accepting input of a first factor that is directly controlled by improving a lifestyle habit of a person subjected to a medical checkup and a second factor that is not directly controlled by improving a lifestyle habit of the person;
generating a lifestyle habit combination pattern;
calculating change amounts of the first factor and the second factor corresponding to the combination pattern; and
predicting a disease risk value representing a disease risk of a disease of the person based on at least the change amount of the second factor.

14. A computer-readable non-transitory recording medium recording a health support program for causing a computer to execute
accepting input of a first factor that is directly controlled by improving a lifestyle habit of a person subjected to a medical checkup and a second factor that is not directly controlled by improving a lifestyle habit of the person,
generating a lifestyle habit combination pattern,
calculating change amounts of the first factor and the second factor corresponding to the combination pattern, and
predicting a disease risk value representing a disease risk of a disease of the person based on at least the change amount of the second factor.
